(19) United States
(12) Patent Application Publication
(10) Pub. No.: US 2021/0128042 A1
(43) Pub. Date: May 6, 2021

(54) SYSTEM FOR CAPTURING BIOSIGNALS

(71) Applicant: MENTALAB GMBH, Berlin (DE)

(72) Inventor: Sebastian HERBERGER, Munich (DE)

(21) Appl. No.: 16/492,087

(22) PCT Filed: Mar. 8, 2017
(86) PCT No.: PCT/EP2017/000309

§ 371 Date: Sep. 6, 2019

(51) Int. Cl. A61B 5/274; A61B 5/257; H01R 13/627

(52) U.S. Cl. CPC A61B 5/274; A61B 5/257; A61B 5/282; A61B 2562/227; H01R 13/6278

(57) ABSTRACT

The present invention relates to a system for detecting biosignals, comprising a sensor unit and a body-attachable patch provided with electrodes and conductor tracks, the sensor unit and the patch being adapted to be mechanically connected to each other, via a connector arranged on the patch, in such a way that an electric connection is established and the sensor unit is simultaneously held via the patch on the body, the sensor unit comprising a housing. According to the present invention, the sensor unit is releasably connectable to the connector by means of a rotation of the housing relative to the connector.

The present invention relates to a system for detecting biosignals, comprising a sensor unit and a body-attachable patch provided with electrodes and conductor tracks, the sensor unit and the patch being adapted to be mechanically connected to each other, via a connector arranged on the patch, in such a way that an electrical connection is established and the sensor unit is simultaneously held via the patch on the body, the sensor unit comprising a housing.

Such systems allow a much more convenient acquisition of biosignals, such as an ECG, since, on the one hand, the position of the electrodes relative to one another is defined by the patch and since, on the other hand, the sensor unit can be worn on the body, whereby in particular long-term measurements will be simplified considerably. Preferably, the sensor unit is held in place on the patient solely by the adhesive force of the patch.

Such systems are known e.g. from US 2015/0164324 A1. According to one embodiment, the connector and the patch are there configured as a disposable, with which the sensor unit is connected for measuring biosignals. The electrical connection of the sensor unit is established with electrical contacts of the connector, which in turn enter into contact with contact areas of the conductor tracks of the patch. The mechanical connection options mentioned are a snap connection, a hook and loop fastener or a screw mechanism.

Another system of this kind is known from EP 1 979 040 B1. The mechanical connection is established by lockingly engaging the sensor unit with arms of the connector, which laterally embrace the housing of the sensor unit. The electrical connection of the sensor unit is established with electrical contacts of the connector, which in turn enter into contact with contact areas of the conductor tracks of the patch. The electrical contacts of the connector may here enter into contact with conductor tracks arranged on a back of the patch, or with conductor tracks arranged on a flap folded under the connector.

It is the object of the present invention to improve the mechanical and/or electrical connection between the sensor unit and the patch.

This object is achieved by the systems according to the independent aspects of the present invention, which will be described in more detail hereinafter.

According to a first independent aspect, the present invention comprises a system for detecting biosignals, comprising a sensor unit and a body-attachable patch provided with electrodes and conductor tracks, the sensor unit and the patch being adapted to be mechanically connected to each other, via a connector arranged on the patch, in such a way that an electrical connection is established and the sensor unit is simultaneously held via the patch on the body, the sensor unit comprising a housing. According to the first aspect, the system is characterized in that the sensor unit is releasably connectable to the connector by means of a rotation of the housing relative to the connector.

This allows the sensor unit to be fixed to the connector by 1-point fixing, which can preferably be established with one hand and which, further preferred, can also be released with one hand. The connection via a rotary movement is also advantageous insofar as a comparatively small base area of the connector will be sufficient for mechanical connection with the housing. This, in turn, increases the wearing comfort. In addition, a connection is obtained that can be established rapidly and intuitively. According to a possible embodiment, the connector comprises a mechanical connection area, which is releasably connectable to a mechanical connection area of the sensor unit by means of a rotary movement.

Preferably, the mechanical connection areas are in locking engagement with each other in at least one defined rotary position. Particularly preferred, the mechanical connection areas are in locking engagement with each other in only one defined rotary position. This ensures reliable electrical contacting, since the respective electrical contact elements are thus spatially assigned to each in an unequivocal manner when they are at the locked position.

According to a possible embodiment, one of the mechanical connection areas comprises at least one projecting annular segment and/or a substantially circular raised area. Alternatively or additionally, the other mechanical connection area may comprise at least one annular groove segment and/or a substantially circular recess.

Preferably, the projecting annular segment and/or the substantially circular raised area is/are, in the connected condition, at least partially accommodated in the annular groove segment and/or the substantially circular recess.

According to a possible embodiment, the mechanical connection areas each surround an electrical connection area, the mechanical connection areas surrounding the electrical connection area preferably substantially circularly. In this way, a reliable electrical connection will be guaranteed by establishing the mechanical connection.

According to a possible embodiment, the mechanical connection areas are adapted to be pushed into one another in at least a first rotary position and to be moved, in the pushed-in condition, to a second rotary position by a rotary movement, the connection areas being locked on one another in the second rotary position. A connection between the sensor unit and the patch can thus be established intuitively. The angle of the rotary movement between the first and the second rotary position may be between 20° and 180°, preferably between 40° and 90°. This makes it easier to establish the connection with only one hand.

According to a possible embodiment, one of the mechanical connection areas comprises at least one guide in which at least one locking element of the other mechanical connection area is guided during a rotary movement.

Preferably, the guide is provided on the mechanical connection area of the connector.

The guide may be configured as a groove in an outer or inner circumference of a mechanical connection area, the groove extending preferably in the circumferential direction.

The guide may have a recess into which the locking element snaps in position at the end of the rotary movement, the locking element being releasable from the snap-in position preferably against the force of a spring.

The recess may, by way of example, be configured as a recess at the end of the groove forming the guide.

In addition, the mechanical connection area carrying the guide may have a recess extending in the direction of the axis of rotation and allowing the locking element to be inserted into the guide.

According to a possible embodiment, the locking element is movable and preferably spring-loaded, and is in particular arranged on the sensor unit in a movable and preferably spring-loaded manner. Due to the spring load, the locking element is preferably pressed against the guide and/or held in a recess of the guide.

Preferably, at least one locking area of the locking element is movable in a radial direction relative to the axis of rotation.

The locking element may be a displaceably supported pin. Preferably, the direction of movement of the pin extends in a radial direction relative to an axis of rotation of the connection between the connector and the sensor unit.

The locking element may be a rotatable hook and/or a rocker. Preferably, the axis of rotation of the hook and/or of the rocker extends parallel to the axis of rotation of the connection between the connector and the sensor unit.

The guide and/or the locking element may be configured such that the torque to be created for the rotary movement increases at least over a subarea of the guide. The user is thus given a haptic feedback and the sensation of a reliable mechanical connection.

This can especially be accomplished by the fact that the distance, which exists between the axis of rotation of the connection between the connector and the sensor unit and the bottom of a groove serving as the guide, changes in the closing direction and becomes larger in the case of a groove arranged on an outer circumference. By way of example, the depth of the groove may decrease.

Preferably, the torque increases continuously over at least 50% of the length of the guide.

According to a possible embodiment, the rotation of the housing takes place about an axis of rotation, which extends at an angle of less than 30° to a normal on the contact plane of the connector with the patch, preferably at an angle of less than 10°, and further preferred perpendicular to the contact plane of the connector with the patch.

According to a possible embodiment, the rotation of the housing for connection between the sensor unit and the connector takes place through an angle of rotation between 20° and 180°, preferably through an angle of rotation between 40° and 90°.

According to a possible embodiment, the sensor unit has at least one operating element, by the actuation of which a locking engagement with the connector can be released.

Preferably, the locking element can be disengaged from the recess against the force of a spring by actuating the operating element.

The operating element may preferably be movably arranged on a housing of the sensor unit, in particular on a lateral or rear area of the housing.

The operating element may be a component separate from the locking element. Alternatively, the operating element and the locking element may be configured as an integral component.

If a spring force is referred to in the context of the present invention, this spring force may be provided e.g. by a separate spring element which preloads two components against each other. Such a separate spring element or a preload is, however, not necessary. A spring force may e.g. also be accomplished by providing between two elements, which are moved relative to each other within the framework of the present invention, a connection of an elastic nature, e.g. an elastic connection between the locking element and/or the operating element and the housing.

According to a second independent aspect, the present invention comprises a system for detecting biosignals, comprising a sensor unit and a body-attachable patch provided with electrodes and conductor tracks, the sensor unit and the patch being adapted to be mechanically connected to each other, via a connector arranged on the patch, in such a way that an electrical connection is established and the sensor unit is simultaneously held via the patch on the body. The system according to the second aspect is characterized in that the connector establishes only the mechanical connection to the sensor unit and that electrical contacting takes place directly between the sensor unit and the patch.

This is advantageous insofar as it is not necessary to provide an electrical contact for the connector. Hence, it can be manufactured e.g. entirely from plastic, for example as an injection-molded part, in particular as a one-piece injection-molded part. The connector can thus be manufactured at a much lower cost.

According to a possible embodiment, the connector is shaped such that at least one contact surface of a conductor track of the patch is accessible from the sensor unit and can in particular be contacted by a contact pin of the sensor unit.

Preferably, the connector has an electrical connection area formed by at least one recess in the connector through which at least one contact surface of a conductor track of the patch is accessible. Preferably, at least one contact pin of the sensor unit may extend through the recess in the connector to the patch.

The recess may be open towards one or a plurality of sides, or it may be surrounded by the connector on all sides.

Preferably, the mechanical connection between the connector and the sensor unit is established on at least two opposed sides of the recess or recesses. This will ensure good electrical contact.

The recess or recesses may be surrounded, e.g. in an annular shape, by the mechanical connection area of the connector.

According to a first variant, a plurality of contact surfaces may be accessible through an recess of the connector. According to a second variant, at least two contact surfaces of the patch may be accessible through separate recesses of the connector.

According to a possible embodiment, the patch side located opposite the connector has arranged thereon a counter element, which supports the patch in the area of the contact points, the counter element being preferably plate-shaped. In particular, the counter element may support the contact points of the patch from behind with respect to contacting with contact pins of the sensor element.

According to a possible embodiment, the patch has a bulge with which it extends into an recess of the connector, through which recess at least one contact surface of a conductor track of the patch is accessible from the sensor unit. In this way, it will suffice when the contact areas, in particular the contact pins, of the sensor unit extend less far into the recess.

The bulge can be created via a raised area on a counter element arranged on the patch side facing away from the connector, the raised area pressing the patch into the opening.

According to a possible embodiment, the sensor element includes an electrical connection area comprising spring-loaded contact pins, which are used for electrically contacting the connector and/or the patch. Due to the spring load on the contact pins, the latter are preferably forced out of the housing and against the contact surfaces of the connector and/or the patch. As a result, reliable contacting will be possible, even if especially the contact points of the patch are of a flexible and/or yielding nature.

Preferably, the contact pins enter directly into contact with contact surfaces of the conductor tracks of the patch, as described above.

Preferably, the spring-loaded contact pins are arranged such that, in the contacted condition, they are at least partially countersunk in the housing of the sensor unit.

According to a possible embodiment, the spring-loaded contact pins project, in the non-contacted condition, beyond a lower edge of the housing of the sensor element. In this way, it will be easier to establish a direct contact with the patch.

According to a variant, the contact points may additionally be reinforced mechanically and/or with respect to their conductivity. This can be done, for example, by applying at least one additional conductive layer.

According to a third independent aspect, the present invention comprises a system for detecting biosignals, comprising a sensor unit and a body-attachable patch provided with electrodes and conductor tracks, the sensor unit and the patch being adapted to be mechanically connected to each other, via a connector arranged on the patch, in such a way that an electrical connection is established and the sensor unit is simultaneously held via the patch on the body, the sensor unit comprising a housing. A first variant of the third aspect is characterized in that the base area of the connector arranged on the patch is a maximum of 70% of the base area of the housing, preferably a maximum of 50%, further preferred a maximum of 30%. A second variant of the third aspect, which may also be provided in combination with the first variant, is characterized in that the mechanical connection with the connector is effected exclusively via a mechanical connection area arranged on the back of the housing, wherein lateral edges of the housing extend preferably at a distance from the mechanical connection area.

The comparatively small base area of the connector makes the latter particularly comfortable to wear on the skin. In particular, it will be possible to compress and/or fold the skin or the patch on the skin under the lateral free sensor unit parts projecting beyond the connector.

Preferably, the housing tapers towards the connector on its back facing the patch. Preferably, the tapering area has a depth of at least 10% of the total depth of the housing, further preferred of at least 20% of the total depth. Alternatively or additionally, the back of the housing facing the patch is convex in shape.

This allows the sensor unit to be fixed to the connector by 1-point fixing, whereby lateral oblique movements of the sensor unit will be possible. This will considerably improve the wearing comfort of the system.

According to a fourth independent aspect, the present invention comprises a system for detecting biosignals, comprising a sensor unit and a body-attachable patch provided with electrodes and conductor tracks, the sensor unit and the patch being adapted to be mechanically connected to each other, via a connector arranged on the patch, in such a way that an electrical connection is established and the sensor unit is simultaneously held via the patch on the body. The fourth aspect is characterized in that the connector is arranged on a patch area configured as a folded flap. Preferably, the mechanical connection between the connector and the patch is here established via the flap.

The arrangement of the connector on the flap has here the advantage that the size and the shape of the non-folded part of the patch can be selected independently of the size and the shape of the connector.

Furthermore, the connector may be arranged on the patch such that it is movable relative to the non-folded part of the patch. In particular, at least a tilting movement may be possible between the connector and the non-folded part of the patch. Preferably, the sensor unit fixed to the connector, and in particular the rigidly connected unit consisting of the sensor unit and the connector, can thus be tilted to the side, if the connector is moved to an inclined position due to folding of the skin, e.g. during movements of the arms and/or the thorax in a supine position. This will improve the wearing comfort.

Alternatively or additionally, the connector may be fixed to the flap alone. However, a counter element of the connector may additionally be fixed to a non-folded part of the patch.

Furthermore, the base area of the connector may be smaller than the base area of the flap. The base area of the connector may come to lie completely on the flap.

Preferably, the flap is movable relative to the non-folded part of the patch. According to a first variant, the flap is arranged on the patch in a freely folded manner. According to a second variant, the flap may, however, also be flexibly fixed and/or point-fixed to an upper surface of the patch on at least one point of the latter. The fixing preferably leaves a distance between the upper surface of the non-folded part of the patch and the flap. The fixing may be releasable according to a possible embodiment. For example, the fixing may by configured as a magnetic contact fastener and/or a hook and loop fastener.

According to a possible further development, a counter element arranged on a flap side facing away from the connector is connected to the non-folded part of the patch.

According to a possible further development, the connector and/or the counter element comprise/comprises a web area around which the flap of the patch is guided in order to prevent a too small buckling radius of the patch. The web area may here be arranged on the connector via one or a plurality of arms. In particular, the flap may be guided around the web area and below the connector, while the arms on both sides of the flap connect the connector to the ends of the web area. According to a preferred embodiment, the distance between the arms may increase outwards, starting from the connector.

According to a possible further development, the folded-over area, via which the flap is connected to the non-folded area of the patch, is wider than the base area of the connector. The weight force of the sensor element is thus distributed over a larger area of the patch.

Alternatively or additionally, the flap may become narrower from the folded-over area, via which it is connected to the non-folded area of the patch, towards its free end.

According to a possible further development, the flap is longer than the non-folded area of the patch to which it is connected, so that a free end of the flap extends beyond an edge of the non-folded area of the patch.

Preferably, the non-folded area extends in a widthwise direction in a band shape.

According to a possible further development, the conductor tracks of the patch are routed from the area of the connector via the flap to the electrodes.

In particular, the conductor tracks may run from the flap in directions opposite to a non-folded, band-shaped area of the patch.

The respective aspects described in more detail hereinbefore may be realized independently of one another. However, the present invention additionally comprises arbitrary combinations of two or of a plurality of independent aspects of the present invention.

Preferred further developments of the present invention, which relate to all the aspects, will be explained in more detail hereinafter.

According to a possible further development, the connector has a maximum diameter of not more than 4 cm, advantageously 3 cm, and/or a substantially circular basic shape.

According to a possible further development, the housing has a maximum diameter of not more than 8 cm, advantageously 6 cm. Furthermore, the housing may have a maximum diameter of at least 2 cm, preferably at least 3 cm.

According to a possible further development, the connector has, starting from the surface of the patch, a maximum depth of 10 mm, preferably a maximum depth of 7 mm.

According to a possible further development, the connector is adhesively attached to the patch. In particular, the connector comprises a contact surface, with which it is adhesively attached to the patch. The contact surface may be substantially annular, by way of example.

Alternatively or additionally, the patch may be clamped in position between the connector and a counter element arranged on the patch side located opposite the connector. The connection between the connector and the counter element may be established e.g. via connection pins extending through the patch.

The sensor unit preferably comprises a communication interface for wireless communication, in particular a radio interface such as Bluetooth and/or WLAN and/or a mobile radio data interface. This allows the wireless transmission of measurement data.

The electrical biosignals, which are detected by the sensor unit, may in particular be an ECG. The latter may in particular be a 1-channel, 2-channel, 4-channel or a 12-channel ECG. For this purpose, the system is preferably attached to the thorax of a person.

According to additional further developments, the system may, alternatively or additionally, be used for EEG measurements (electroencephalography) and/or EOG measurements (electrooculography). In these cases, the system may be attached to the patient's head. According to another variant, the system may be used for EMG (electromyography) measurements. In this case, the system may be placed over an arbitrary muscle of the patient, whose activity can be measured.

The patch preferably comprises a plurality of electrodes, preferably more than two electrodes.

The patch preferably comprises an adhesive layer on its side facing the body, the adhesive layer being preferably covered by a peelable protective liner.

The connector may preferably be adapted to be directly connected to the housing of the sensor unit. In particular, the mechanical connection area of the sensor unit may here be formed by the housing.

The connector is preferably fixedly connected to the patch, i.e. a separation of the patch from the connector is not intended within the framework of normal handling.

The connection between the patch and the connector may configured to be splash-proof and/or waterproof, in particular by means of an adhesive joint.

Furthermore, the connection of sensor unit, connector and patch may be configured to be splash-proof and/or waterproof. This is advantageous insofar as patients and/or users can take a shower with it, and/or sweat will not cause any artifacts.

The patch and the connector arranged on the patch preferably form a disposable.

The present invention also comprises the respective sensor unit of the above described systems according to the independent aspects of the present invention and the above described preferred further developments.

In addition, the present invention comprises the respective connector of the above described systems according to the independent aspects of the present invention and the above described preferred further developments.

In addition, the present invention comprises the respective patch, with the connector arranged on the patch, of the above described systems according to the independent aspects of the present invention and the above described preferred further developments.

The present invention further comprises, independently of as well as in combination with the above representation, the following aspects:

1. A system for detecting biosignals, comprising a sensor unit and a body-attachable patch provided with electrodes and conductor tracks, the sensor unit and the patch being adapted to be mechanically connected to each other, via a connector arranged on the patch, in such a way that an electrical connection is established and the sensor unit is simultaneously held via the patch on the body, the sensor unit comprising a housing,

characterized in that

the sensor unit is releasably connectable to the connector by means of a rotation of the housing relative to the connector.

2. The system according to aspect 1, wherein the connector comprises a mechanical connection area, which is releasably connectable to a mechanical connection area of the sensor unit by means of a rotary movement,
wherein, preferably, the mechanical connection areas are in locking engagement with each other in at least one defined rotary position and, further preferred, in only one defined rotary position,
and/or wherein, preferably, one of the mechanical connection areas comprises at least one projecting annular segment and/or a substantially circular raised area and/or the other mechanical connection area comprises at least one annular groove segment and/or a substantially circular recess, the projecting annular segment and/or the substantially circular raised area being, in the connected condition, preferably accommodated, at least partially, in the annular groove segment and/or the substantially circular recess,
and/or wherein, preferably, the mechanical connection areas each surround an electrical connection area, the mechanical connection areas surrounding the electrical connection area preferably substantially circularly.

3. The system according to aspect 2, wherein the mechanical connection areas are adapted to be pushed into one another in at least a first rotary position and to be moved, in the pushed-in condition, to a second rotary position by a rotary movement, the connection areas being locked on one another in the second rotary position,
and/or wherein one of the mechanical connection areas comprises at least one guide in which at least one locking element of the other mechanical connection area is guided during a rotary movement,
wherein the guide is preferably provided on the mechanical connection area of the connector,
and/or wherein the guide is preferably configured as a groove in an outer or inner circumference of the mechanical connection area, the groove extending preferably in the circumferential direction,
and/or wherein the guide preferably has an recess into which the locking element snaps in position at the end of the rotary movement, the locking element being releasable from the snap-in position preferably against the force of a spring, the recess being preferably configured as a recess at the end of a groove forming the guide,
and/or wherein the mechanical connection area carrying the guide has an recess extending in the direction of the axis of rotation and allowing the locking element to be inserted into the guide.

4. The system according to aspect 3, wherein the locking element is movable and preferably spring-loaded, and is in particular arranged on the sensor unit in a movable and preferably spring-loaded manner, wherein, preferably, at least one locking area of the locking element is movable in a radial direction relative to the axis of rotation, and/or wherein the locking element is preferably a displaceably supported pin and/or a rotatable hook and/or a rocker, wherein the direction of movement of the pin preferably extends in a radial direction relative to an axis of rotation of the connection between the connector and the sensor unit, and/or the axis of rotation of the hook and/or of the rocker preferably extends parallel to the axis of rotation of the connection between the connector and the sensor unit.

5. The system according to one of the aspects 3 or 4, wherein the guide and/or the locking element are configured such that the torque to be created for the rotary movement increases at least over a subarea of the guide, in particular by reliably changing, in the closing direction, the distance between the axis of rotation of the connection between the connector and the sensor unit and the bottom of a groove serving as the guide, the torque preferably increasing continuously over at least 50% of the length of the guide.

6. The system according to one of the preceding aspects, wherein the rotation of the housing takes place about an axis of rotation, which extends at an angle of less than 30° to a normal on the contact plane of the connector with the patch, preferably at an angle of less than 10°, and further preferred perpendicular to the contact plane of the connector with the patch, and/or wherein the rotation of the housing for connection between the sensor unit and the connector takes place through an angle of rotation between 20° and 180°, preferably through an angle of rotation between 20° and 90°.

7. The system according to one of the preceding aspects, wherein the sensor unit has at least one operating element, by the actuation of which a locking engagement with the connector can be released, wherein, by actuating the operating element, the locking element can preferably be disengaged from the recess against the force of a spring, and/or wherein the operating element is preferably movably arranged on a housing of the sensor unit, in particular on a lateral or rear area of the housing, and/or wherein the operating element is preferably a component separate from the locking element, or wherein the operating element and the locking element are configured as an integral component.

8. A system, in particular a system according to one of the preceding aspects, for detecting biosignals, comprising a sensor unit and a body-attachable patch provided with electrodes and conductor tracks, the sensor unit and the patch being adapted to be mechanically connected to each other, via a connector arranged on the patch, in such a way that an electrical connection is established and the sensor unit is simultaneously held via the patch on the body,
characterized in that
the connector establishes only the mechanical connection to the sensor unit and that electrical contacting takes place directly between the sensor unit and the patch.

9. The system according to aspect 8, wherein the connector is shaped such that at least one contact surface of a conductor track of the patch is accessible from the sensor unit, wherein the connector preferably has an electrical connection area formed by at least one recess in the connector through which at least one contact surface of a conductor track of the patch is accessible, wherein preferably a plurality of contact surfaces are accessible through an recess of the connector and/or wherein preferably at least two contact surfaces of the patch are accessible through separate recesses of the connector,
and/or
wherein the patch side located opposite the connector has arranged thereon a counter element, which supports the patch in the area of the contact points, the counter element being preferably plate-shaped,
and/or wherein the patch has a bulge with which it extends into an recess of the connector, through which recess at least one contact surface of a conductor track of the patch is accessible from the sensor unit, wherein the bulge is created preferably via a raised area on a counter element arranged on the patch side facing away from the connector, the raised area pressing the patch into the opening.

10. The system according to one of the preceding aspects, wherein the sensor element includes an electrical connection area comprising spring-loaded contact pins, which are used for electrically contacting the connector and/or the patch and which preferably enter directly into contact with contact surfaces of the conductor tracks of the patch, wherein the spring-loaded contact pins are arranged such that, in the contacted condition, they are preferably at least partially countersunk in the housing of the sensor unit and/or wherein, in the non-contacted condition, the spring-loaded contact pins preferably project beyond a lower edge of the housing of the sensor element.

11. A system, in particular a system according to one of the preceding aspects, for detecting biosignals, comprising a sensor unit and a body-attachable patch provided with electrodes and conductor tracks, the sensor unit and the patch being adapted to be mechanically connected to each other, via a connector arranged on the patch, in such a way that an electrical connection is established and the sensor unit is simultaneously held via the patch on the body, the sensor unit comprising a housing,
characterized in that
the base area of the connector arranged on the patch is a maximum of 70% of the base area of the housing, preferably a maximum of 50%, further preferred a maximum of 30%, and/or wherein the mechanical connection with the connector is effected exclusively via a mechanical connection area arranged on the back of the housing, wherein lateral edges of the housing extend preferably at a distance from the mechanical connection area.

12. The system according to aspect 11, wherein the housing tapers towards the connector on its back facing the patch, wherein the tapering area preferably has a depth of at least 10% of the total depth of the housing, further preferred of at least 20% of the total depth, and/or wherein the back of the housing facing the patch is preferably convex in shape.

13. A system, in particular a system according to one of the preceding aspects, for detecting biosignals, comprising a sensor unit and a body-attachable patch provided with electrodes and conductor tracks, the sensor unit and the patch being adapted to be mechanically connected to each other, via a connector arranged on the patch, in such a way that an electrical connection is established and the sensor unit is simultaneously held via the patch on the body,
characterized in that
the connector is arranged on a patch area configured as a folded flap, and is preferably movable relative to the non-folded part of the patch.

14. The system according to aspect 13, wherein, at least a tilting movement is preferably possible between the connector and the non-folded part of the patch,
and/or
wherein the flap is movable relative to the non-folded part of the patch, wherein, preferably, the flap is arranged on the patch in a freely folded manner or is flexibly fixed and/or point-fixed to an upper surface of the patch on at least one point of the latter, the fixing preferably leaving a distance between the upper surface of the non-folded part of the patch and the flap,
and/or
wherein a counter element arranged on a flap side facing away from the connector is connected to the non-folded part of the patch,
and/or wherein the connector and/or the counter element comprise/comprises a web area around which the flap of the patch is guided in order to prevent a too small buckling radius of the patch,
and/or wherein the folded-over area, via which the flap is connected to the non-folded area of the patch, is wider than the base area of the connector and/or wherein the flap decreases in width from the folded-over area, via which it is connected to the non-folded area of the patch, towards its free end,
and/or wherein the flap is longer than the non-folded area of the patch, to which it is connected, so that a free end of the flap extends beyond an edge of the non-folded area of the patch, the non-folded area preferably extending in a widthwise direction in a band shape,
and/or wherein the conductor tracks of the patch are routed from the area of the connector via the flap to the electrodes, wherein, preferably, conductor tracks run from the flap in directions opposite to a non-folded, band-shaped area of the patch.

15. The system according to one of the preceding aspects, wherein the connector has a maximum diameter of not more than 4 cm, advantageously 3 cm, and/or a substantially circular basic shape, and/or wherein the housing has a maximum diameter of not more than 8 cm, advantageously 6 cm, and/or wherein the housing has a maximum diameter of at least 2 cm, advantageously at least 3 cm, and/or wherein the connector has, starting from the surface of the patch, a maximum depth of 10 mm, preferably a maximum depth of 7 mm.

16. The system according to one of the preceding aspects, wherein the connector is adhesively attached to the patch and/or wherein the patch is clamped in position between the connector and a counter element arranged on the patch side located opposite the connector.

17. A sensor unit for a system according to one of the preceding aspects.

18. A connector or a patch with a connector for a system according to one of the preceding aspects.

The present invention according to one or a plurality of the above described aspects can have in particular one or a plurality of the following advantages:
In acute situations and/or for emergency use, a mobile, easy-to-install solution for measuring an ECG (as 1-channel or 12-channel) represents a previously unattainable advantage.
In the monitoring of patients in a hospital, the present solution means that patients will be mobile and no longer “bound” to the monitor by a cable. This is advantageous insofar as nursing activities, such as washing or going to the bathroom, can also take place under monitoring. In particular when going to the toilet, patients frequently collapse or become unconscious—a situation in which, previously, there was often a pause in monitoring.
The absence of cables leads to improved wearing comfort, but also to a better signal quality (no cable movement artifacts and better electrode stability, no tearing off).
All the elements that are in contact with the body are disposable. This leads to significantly improved hygienic aspects in comparison with conventional ECGs, where the systems consecutively come into contact with hundreds of patients.
A system by means of which a patient will also be able to carry out a measurement independently and which allows a transmission of the data by radio, allows a decentralized measurement on the part of the patient, who will be able to execute the measurement independently, if necessary. This will be of advantage, e.g. for intermittently occurring cardiac arrhythmia or for “chest pain”, i.e. angina pectoris.

The present invention will now be described in more detail on the basis of embodiments and drawings, in which

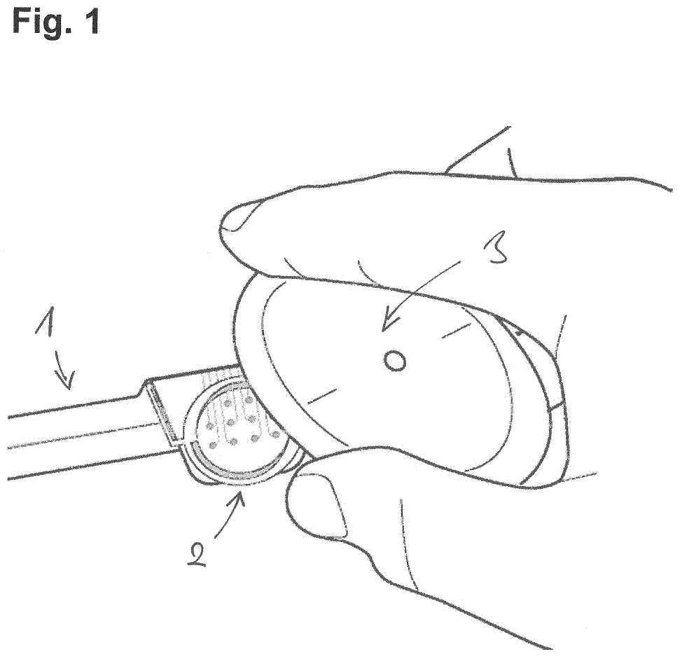

FIG. 1 shows an embodiment of a system according to the present invention, comprising a patch with a connector and a sensor unit, in a perspective view in which the sensor unit is not yet connected to the connector,

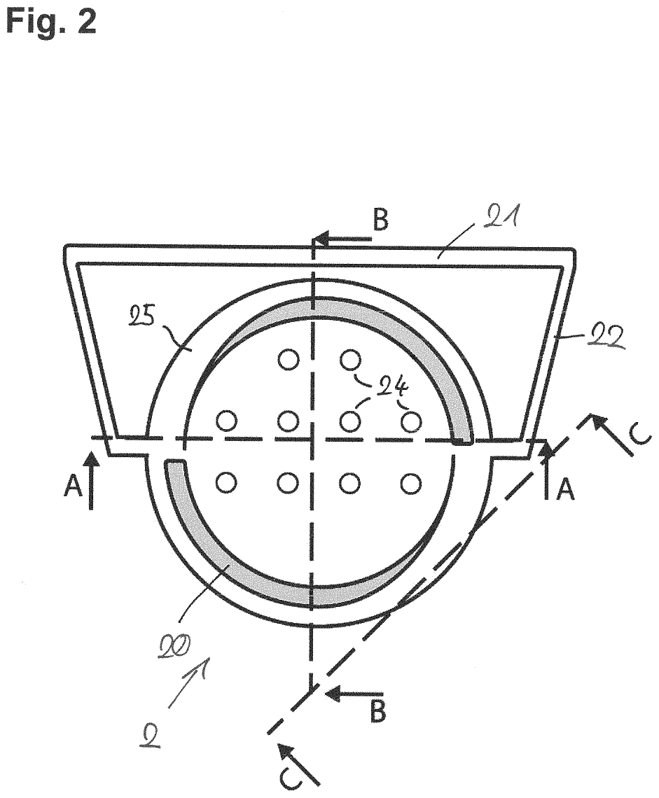

FIG. 2 shows a top view of a first embodiment of a connector,

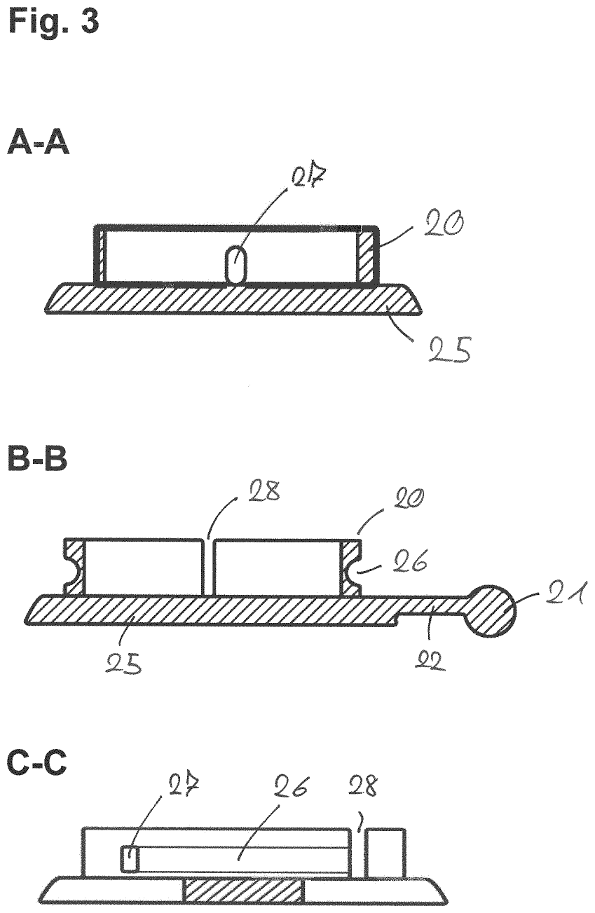

FIG. 3 shows three sectional views through the embodiment of a connector shown in FIG. 2,

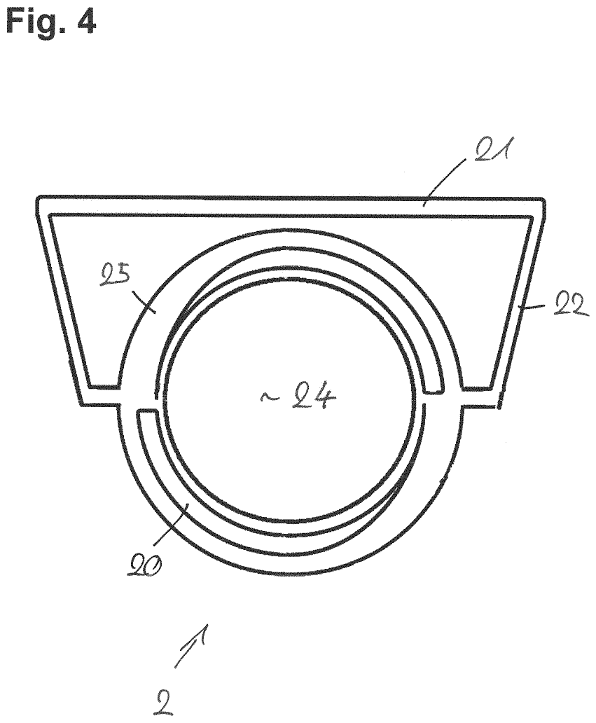

FIG. 4 shows a top view of a second embodiment of a connector,

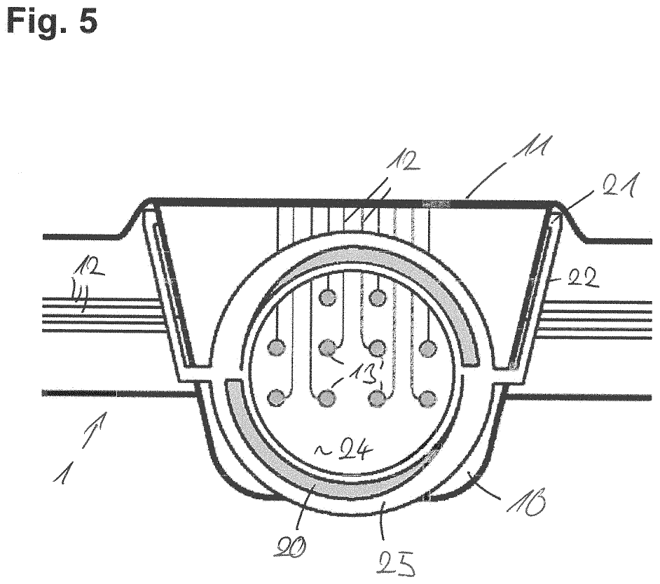

FIG. 5 shows a top view of the second embodiment of a connector attached to a flap of the patch in the present embodiment,

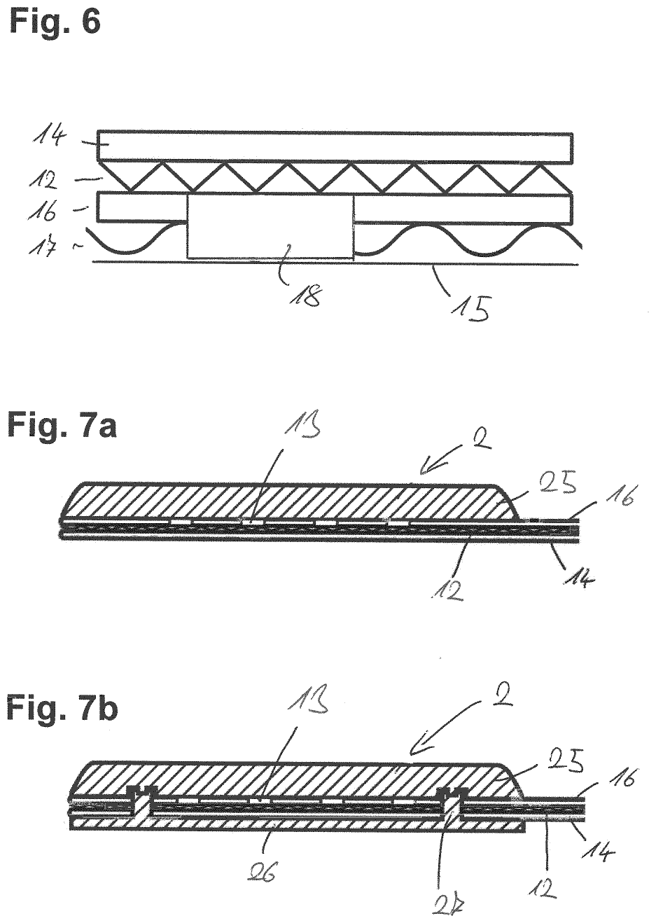

FIG. 6 shows a sectional view through an embodiment of the patch perpendicular to the patch plane in an area with an electrode,

FIG. 7a shows a sectional view through the embodiment of the patch perpendicular to the patch plane with a connector adhesively attached to the patch,

FIG. 7b shows a sectional view through the embodiment of the patch perpendicular to the patch plane with a connector attached to the patch via a counter element,

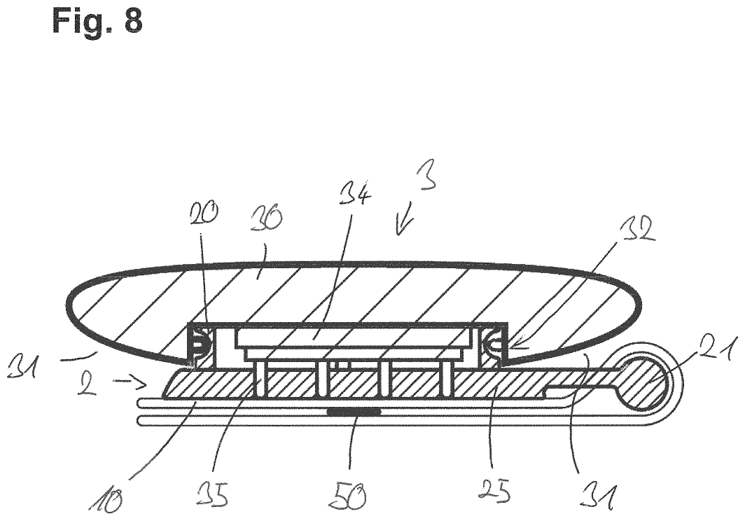

FIG. 8 shows, perpendicular to the patch plane, a sectional view of the embodiment of a system according to the present invention, comprising a patch with a connector and a sensor unit, the sensor unit being connected to the connector, and

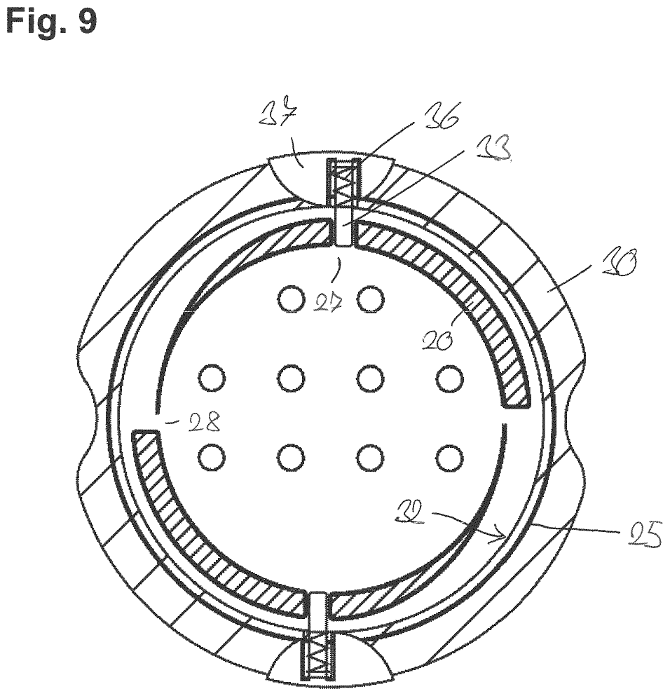

FIG. 9 shows a sectional view through the embodiment shown in FIG. 8, parallel to the patch plane on the level of the mechanical connection areas of the sensor unit and the connector.

The embodiment of the present invention described in FIGS. 1 to 9 is a mobile system that can be worn on the body and is used for recording and transmitting, preferably wirelessly, biosignals. The embodiment realizes all the independent aspects of the present invention in combination. The preferred further development of the respective independent aspects of the present invention explained in more detail hereinafter on the basis of the embodiment is, however, also part of the present invention when seen individually and without the other aspects.

The present embodiment of the system according to the present invention shown in FIG. 1 consists of a measuring device in the form of a wireless sensor unit 3 and a patch 1, which is attached to the skin of the test person. The patch 1 comprises electrodes 18 for deriving biosignals and electrical conductor tracks 12, which transmit the biosignals (ExG) from the electrodes 18 to the measuring device 3. The patch 1 is configured to be applied to the patient for use and disposed of after use.

The sensor unit 3 is attached to the patch 1 via a connector 2 and held in place. The connector 2 is configured such that the sensor unit 3 can very easily be attached to the patch 1, preferably as a rotary movement carried out with one hand. The connector 2 ensures that, when in use, the sensor unit 3 will be held mechanically on the patch 1 for a long period of time, i.e. the sensor unit 3 is rendered wearable on the body.

In addition, the mechanical connection established by the connector 2 between the sensor unit 3 and the patch 1 establishes simultaneously also an electrical connection for transmitting the biosignals from the patch 1 to the sensor unit 3. On the side of the sensor unit 3, the contacts may be realized e.g. in the form of spring-loaded contact pins 35 (so-called spring-PINs), which protrude from the sensor unit 3 and which, preferably, enter into direct contact with the conductors 12 of the patch.

A primary field of use of the system is the measurement, recording and wireless transmission of medical biosignals and of data in medical diagnostics, monitoring and treatment. The data may be transmitted to mobile terminal (smartphones, tablets, computers), via radio nodes to servers, or directly via a mobile radio and/or satellite network to databases and/or servers. The data may be evaluated by specialists or attending physicians directly on the patient, or evaluated in real time in evaluation centers, or stored for future evaluation. Further possibilities of use for the system are the fields of sports and “wellness”.

Preferred features of the embodiment, which can be realized separately as well as in combination, will first be explained briefly hereinafter on the basis of the three components patch 1, connector 2 and sensor unit 3:

### Patch

The electrodes 18 and the conductors 12 are integrated in the patch 1. The application of the patch will thus intuitively always lead to a correct positioning of the electrodes 18.

The patch 1 has a folded flap 10 on which the connector 2 is arranged. This allows a very narrow design of the areas of the patch 1 adhering to the skin of the test person and/or an increased movability of the sensor unit 3 relative to the patch 1.

The fact that the patches 1 are used only once (“disposable”) makes the system hygienically superior to previous solutions, since all the parts that are in contact with the patient are disposed of after use.

### Connector

The connector 2 allows a connection to be established with one hand and/or is connected to the sensor unit via a rotary mechanism.

When the sensor unit 3 is correctly attached to the connector 2, a haptic feedback will preferably occur during engagement and/or when the connection has been established successfully (“click”).

The connector 2 only establishes the mechanical connection and leaves contact areas 13 of the conductor tracks 12 accessible for direct contacting with the sensor unit 3, in particular through one or more recesses 24.

The connector has a simple structural design and may be manufactured as an injection molded part. This reduces the cost of the disposables, which consist of connector and patch.

### Sensor Unit

The housing 30 of the sensor unit 3 is designed to be comfortable to wear, preferably with a shape of the housing back 31 that narrows towards a mechanical connection area with the connector.

The electrical contact is established directly with the patch, in particular via spring-loaded contact pins 35 (so-called spring-PINs) protruding beyond the sensor unit.

In the following, the signals that can be detected through the embodiment of the system and the possibilities of using the system will be described in an overview:

### Signals

At least one and preferably a plurality of the following signals can be recorded directly as raw data making use of the embodiment of the system. From this, further parameters can be derived and calculated:
ECG
EEG
EOG
EMG

### Possible Applications

The system preferably allows at least one and preferably a plurality of the possible applications following hereinafter:

a. 12-channel (or 16-channel) ECG, (e.g.) in a routine checkup and/or in the case of chest pain and/or unclear abdominal or thoracic complaints.

b. Exercise ECG (especially also for use in a mobile environment—e.g. jogging, hiking, rowing, etc.).

c. Long-term ECG (e.g. in the case of suspected cardiac arrhythmias) or long-term EEG (with an application time of days or weeks, e.g. in the case of suspected epilepsy).

d. as telemetry solution and/or home-care ECG, applicable by the patient himself.

e. Monitoring of ECGs and of other vital parameters of patients in an ambulance and/or patient transport and/or in hospital and/or in intensive care.

f. as acute EEG (e.g. in the case of an unclear reduction of vigilance, exclusion of epilepsies, resuscitation of patients for rhythm analysis).

g. Permanent home monitoring in the case of life-threatening arrhythmias and/or for the diagnosis of cardiac arrhythmias in acute cases or “remote”.

h. for scientific examinations and/or biosignal measurements.

The sensor unit preferably has at least one interface for wireless transmission of data, in particular a radio interface, in particular for near-field communication such as Bluetooth (2.0, 4.0/smart, or 5.0), WLAN and/or NFC and/or a mobile radio data interface, e.g. via LTE, UMTS and/or GSM. The sensor unit may additionally have a wired interface for data transmission, e.g. a USB interface.

In a first variant, the biosignals can be transmitted as raw data. In a second variant, the biosignals can be evaluated by the sensor unit and data can be transmitted on the basis of the evaluation.

For transmitting and/or processing the biosignals, one or a plurality of the solutions following hereinafter may be implemented.

a.) Transmission of biosignals from the sensor unit via a wireless interface (e.g. Bluetooth (2.0, 4.0/smart, or 5.0), WLAN, NFC or LTE) to a mobile terminal and/or computer (direct visualization) from there via a wireless interface (LTE, GSM, Internet) to a server and/or a database and/or a computer center (from where they can be retrieved onto mobile devices and/or via the Internet).

b.) Transmission of biosignals from the sensor unit via a wireless interface (e.g. WLAN and/or LTE and/or GSM and/or Internet and/or satellite) to a server and/or a database and/or a computer center (from where they can be retrieved onto mobile devices and/or via the Internet)

c.) Pattern recognition and/or signal analysis running in the sensor unit. If an abnormal pattern (e.g. arrhythmias and/or epilepsy) is detected, an alarm and/or a message is sent to the patient's mobile phone and/or the physician and/or a data center

d.) from the sensor unit via a wireless interface (e.g. Bluetooth (2.0 or 4.0/smart, 5.0), WLAN, NFC or LTE) (optionally also via a mobile terminal and/or a computer) to an in-house patient data management system (e.g. KIS, SAP, or the like) in a hospital or a medical practice

e.) recording and storage of data in the sensor unit for future transmission to a computer (via cable and/or wireless interface (e.g. mobile network and/or WLAN)) and evaluation (e.g. as long-term ECG)

In the following, the components and aspects of the present invention will be described again in detail on the basis of the embodiment:

### Sensor Unit

The sensor unit comprises one, a plurality of and preferably all the following components
battery and/or accumulator
charging circuit
signal processing module for the individual biosignal channels (e.g. filter, integrated AD converter front end, amplifier)
memory (e.g. flash, RAM)
micro-USB socket for electrically charging and/or for transmitting data
processor (e.g. ARM Cortex)
wireless radio interface (e.g. BT 2.0 and/or 4.0 and/or 5.0 and/or WLAN GSM)
LED and/or a plurality of LEDs as status indicators
housing

As shown in the sectional view in FIG. 8, the housing 30 of the sensor unit 3 has on its back 31 a mechanical connection area 32 including the connector 2. The base area of the sensor unit, i.e. its maximum surface area in a plane parallel to the patch plane, is larger than the base area of the mechanical connection area 32 and/or of the connector 2. The mechanical connection with the connector is established exclusively with the mechanical connection area 32, which is arranged on the back 31 and which is surrounded on all sides by surface sections of the back 31 of the housing. The surface sections are preferably inclined in a forward direction from the connector. In particular, the surface sections form a convex area without any edge areas.

The size ratios and/or the position of the connection and/or the shape of the back lead to an increase in wearing comfort, since the housing allows a swiveling movement relative to the patch. This leads to a higher degree of freedom as regards wearing of the connector, since there is only one contact point between the sensor unit and the patch and since, laterally of the contact point, a clearance remains between the patch and the back of the housing. In addition, due to the convexity of the back of the sensor unit, edges that could be pressed against the body will be avoided.

Due to the comparatively small connector and/or its arrangement on the flap, the patch has only small rigid areas or no rigid areas at all. The patch can thus fold and/or bend with the body surface when the test person moves. This will improve the wearing comfort significantly.

According to a possible embodiment, the housing of the sensor unit may be configured to be splash-proof and/or waterproof. The housing may consist of at least two housing halves, the connection area between the two housing halves being provided with a seal.

### Patch

The structural design of the patch is shown in FIG. 6. The patch comprises the following components:
carrier substrate 14 (e.g. PET film, thickness e.g. 100 micrometers)
adhesive 17 on the lower surface of the patch, through which the patch is attached to the skin
electrical conductors 12, preferably printed onto the carrier substrate, e.g. from ink containing Ag and/or AgCl or carbon particles (carbon)
electrodes 18 integrated in the patch and consisting e.g. of a layer of hydrogel applied on top of the conductor track, the layer of hydrogel containing e.g. ions (e.g. NaCl) and transmitting the biosignals from the body to the conductors.
the open part of the conductor below the hydrogel may optionally be chlorinated before the hydrogel is applied
a protective liner 15 that covers the electrode areas and the adhesive until use
a packaging in which the patch can be stored for a long time (for example, a packaging unit made of a plastic film or a metallized film)

Production of the Patch:

Provision of the Carrier Material and of the Conductor Tracks
the carrier material 14 is printed-on to provide the conductor tracks 12, e.g. printed-on as a sheet or in roll form by means of screen printing and/or flexo printing processes
to this end, a conductive ink (containing Ag or carbon, by way of example) is first printed onto one side of the carrier material 14 and sintered (by exposure and/or drying, etc.)
the parts of the carrier material 14 that are not electrode sites in skin contact and are not used as contact areas 13 for electrical contacting with the sensor unit are covered with a protective layer 16 (e.g. a biocompatible polymer)
the protective layer 16 has applied thereto an adhesive material 17, which attaches the patch to the patient's skin. The adhesive material forms a bonding layer.
optionally, the protective layer 16 and the bonding layer 17 may also be provided by the same material

Manufacture of the Electrodes:
possibly, the conductive ink is chlorinated at the locations which are free of protective material 16 and adhesive 17 and which represent the skin electrodes 18, or a chloride-containing and/or chlorinated conductive ink is printed on top of the first ink
a hydrogel or a liquid containing chloride ions is applied to the optionally chlorinated electrode sites. This is done, for example, by manually placing an already finished hydrogel or by mechanical deposition of a liquid hydrogel.

Establishing the Contact Areas:

At the locations which are intended to form the contact areas 13 of the conductor tracks 12, the conductor tracks are flat in shape. Furthermore, there is no protective material 16 or adhesive 17 in the area of the contact areas 13. The protective material 16 and the adhesive 17 may have provided therein a separate recess for each contact area 13 or a common recess for all the contact areas 13.

According to a variant, the contact areas 13 may be reinforced mechanically and/or with respect to their conductivity. This can be done e.g. by applying an additional conductive layer, consisting e.g. of a conductive plastic, and/or by using several layers of conductive ink, e.g. by overprinting the contact areas with one or a plurality of further layers of the ink.

Protective Liner:

The patient-side surface, which now comprises an adhesive material 17 and electrodes 18, has applied thereto a protective liner 15, which protects against aging, drying out and physical damage and is removed before use. (comparable to a conventional adhesive plaster)

Surface:

The non-patient-side surface of the carrier material 14 has optionally applied thereto an additional layer of a soft and/or surface-structuring material (e.g. textile or textile-like). Alternatively, this surface may already be arranged on the initial carrier material.

Attaching the Connector to the Patch:

FIG. 5 and FIGS. 7 a/b and 8 show how the connector is attached to the patch:

A part of the patch consists of a flap 10, i.e. a bulge or a lateral protuberance, on which all the conductor tracks 12 end in a specific configuration in contact areas 13, in particular contact surfaces. These contact areas 13 are the ends of the printed conductor tracks 12. By folding over the flap 10, the free ends of the conductor tracks 12 come to lie in an exposed condition on the patch side, which is in principle the side facing the patient, but which, due to folding over, has now become the side facing away from the patient. At this location of the patch, the connector 12 is applied to the patch side, which is in principle the side facing the patient, but which, due to folding over, has now become the side facing away from the patient. The connector is in particular a plastic part. It comprises one or a plurality of recesses 24 through which the contact areas 13 of the conductor tracks 12 are accessible.

The bending radius of the patch is limited by the flexibility of the printed ink 12. The latter should normally not be less than a minimum bending radius of e.g. 2 mm. Therefore, the folding point 11 of the connector flap 10, at which a 180° fold is executed, is guided over a web area 21, which is configured as part of the connector in the present embodiment. The web area 21 may, for example, have the shape of a round, horizontal cylinder with a diameter of 3-4 mm, and prevents the flap 10 from being excessively folded. The web area 21 may be connected to the rest of the connector via connecting arms 22. The lug 10 is guided around the web area 21 and below the connector, cf. FIG. 8. The connecting arms 22 preferably extend from the connector at an oblique angle away from each other to the web area 21, cf. FIGS. 2, 4 and 5. The web area may also be arranged in the same way on a counter element 26 of the connector 2.

The conductors 12 in the patch serve the purpose of conducting the biosignals from the electrodes 18 on the skin through the patch to the connector 2, where they are transmitted directly to the sensor unit via the contact areas 13.

The flap 10 increases in width from its free end to the folding area 11. The weight of the sensor unit is thus distributed over a wider area of the patch.

The flap 10 has, from its free end to the folding area 11, a greater length than the area of the patch to which it is attached, and thus extends beyond the latter with its free end. In this way, the patch can be configured as a narrow band in the area in which it is adhesively attached to the patient.

Due to the fact that the connector is attached to the flap, the connector is movable relative to the patch areas adhesively attached to the skin.

The flap may either be folded over freely, or, as shown in FIG. 8, be connected on its back to the surface of the patch. This takes preferably place via a punctiform fastening area 50 having a certain height. As a result, movement is still possible between the patch and the flap and thus between the patch and the connector. According to a possible embodiment, the fastening may be releasable and may, in particular, be provided via a magnetic connection.

Connector

The connection between the sensor and the patch is secured by the connector, a first embodiment of which is shown in FIG. 2 and a second embodiment of which is shown in FIG. 4.

The connector is, in both embodiments, a mechanical connection element, made e.g. of a biocompatible polymer, that is attached to the patch side, which is in principle the side facing the patient, but which, due to folding over, has now become the side facing away from the patient. The dimensions of the connector are e.g. 2-5 cm in diameter and 3-5 mm in height.

The connector 2 is adhesively attached to the patch, cf. FIG. 7a, and/or held by a counter element 26 on the patch side, which is in principle the side facing away from the patient, but which, due to folding over, has now become the side facing the patient, cf. FIG. 7b. For establishing a connection between the connector 2 and the counter element 26, webs 27 are provided, which extend through the patch or extend past the edge of the patch on the outside thereof and which, for example, enter into locking engagement with the other element and/or form a snap-on fastening therewith. The counter element 26 may be a plate having e.g. a thickness between 0.5 mm and 2 mm, in particular a thickness of 1 mm. The counter element may be made of a biocompatible plastic or of cardboard. In both cases, the connector rests with the lower surface of its base plate 25 on the patch side which is in principle the side facing away from the patient, but which, due to folding over, has now become the side facing the patient.

The connector 2 establishes the mechanical connection between the sensor unit 3 and the patch 1, i.e. it fastens the sensor unit 3 to the patch 1. The mechanical connection of the sensor unit 3 to the patch 1 established by means of the connector 2 defines the position of the sensor unit on the patch, and thus the position of the contacts, in particular the position of the contact pins 35 of the sensor unit 3 relative to the contact areas 13 of the conductor tracks 12 on the folded flap 10, cf. FIG. 4. This ensures that the contact pins 35 of the sensor unit are correctly positioned and contacted.

As can especially be seen from FIG. 8, the contact areas 13 of the conductors 12 of the patch are contacted directly by the contact pins 35 of the sensor unit. To this end, the connector has one or more recesses 24 in the area of the contact areas 13, through which the contact pins 35 can pass and enter into contact with the contact areas 13 on the patch.

In this respect, the connector may have provided therein a separate recess 24 for each contact area 13, as shown in FIG. 2 in the case of the first embodiment. According to a preferred embodiment, however, the connector only has one recess 24, which is common to all the contact areas 13 and which is configured e.g. as an opening in a base plate 25 of the connector, with which the connector rests on the patch. This structural design is shown in the case of the second embodiment in FIG. 4. In this case, the connector preferably has the shape of a ring surrounding the electrical contact areas 13 of the patch. As for the rest, the two embodiments in FIGS. 2 and 4 are identical.

In order to allow the contact areas 13 of the patch to be more easily contacted by the contact pins 35 of the sensor unit, the connector may comprise a counter element 26 of the type shown in FIG. 7b. The patch may rest on the counter element, at least in the area of the contact areas 13 of the patch, so that the contact force of the contact pins 35 on the patch will be increased and buckling of the patch in this area will be avoided. According to a first variant, the counter element may be plate-shaped with a flat surface in the area of the contact areas 13 of the patch. According to a second embodiment, the counter element may have a raised area on its surface in the area of the contact areas 13 of the patch, through which the contact areas are pressed into the recess 24 in the direction of the sensor unit. Hence, it will suffice when the contact pins of the sensor unit protrude to a lesser extent from the housing 30 of the sensor unit in order to enter into contact with the contact areas 13.

For the user, the connector 2 serves to easily and intuitively attach the sensor unit 3 to the patch 1. The use of one hand will be sufficient to attach the sensor unit to the connector. The structural design of the connector allows the latter to be attached via haptic-intuitive elements, so that it can even be attached without direct visual contact.

In the present embodiment, the connector is configured such that attaching the sensor unit 3 will be accomplished by a rotary movement, e.g. clockwise. This rotary movement may comprise a rotation angle of 20-180°. First, the sensor unit 3 is positioned at a defined, preferably marked first rotary position. The markings may be defined either optically, e.g. by lines, or mechanically, e.g. by surface structures and/or curvatures, and allow the user to clearly position the sensor unit at the first position on the connector.

The structural design of the connector is shown in more detail in FIGS. 2 and 3. The connector has a base plate 25, with which it is arranged on the surface of the patch. The base plate has provided thereon a projecting annular area 20, which defines the mechanical connection area for connection to the sensor unit 3. The outer circumference of the annular area 20 has provided therein a circumferentially extending guide 26 in the form of a groove. At the end of the guide 26, a recess 27 is provided as a locking device. Furthermore, axially extending recesses 28 are provided, which lead to the beginning of the circumferentially extending guide.

The base plate 25 has additionally provided thereon arms 22, on which the web area 21 is arranged.

The mechanical connection to the sensor unit can be seen in FIGS. 8 and 9. The lower surface of the sensor unit has provided thereon two radially inwardly directed, spring-loaded locking elements 33, in the present embodiment in the form of horizontally arranged pins, which are first inserted in the first position in an axial direction into the connector recesses 28 provided for this purpose. The locking elements 33 are thus located at the beginning of a guide 26 provided on the outer circumference of the connector 2. After insertion into the guide 26 in position 1, the locking elements are moved by the rotary movement up to and into to a second rotary position around the connector. At the end of rotation, upon reaching the second rotary position, the locking elements 33 lock in the second rotary position by entering into locking engagement with recesses 27, in particular recesses and/or depressions and/or cutouts on the outside of the connector. When the locking elements 33 enter into locking engagement with these recesses 27, they provide a haptic feedback to the user. In the second rotary position, the electrical contacts come to lie on one another in the correct position, so that the electrical connection is provided.

Optionally, the guide 26 for the locking elements 33 in the connector may be configured such that the rotary movement will gradually appear more challenging for the user. This creates the impression of winding a spring, which is an improvement as regards the haptic sensation of the user. This is accomplished mechanically by gradually increasing the distance between the bottom of the guide 26, on which the locking elements 33 slide, and the axis of rotation. According to the embodiment, the annular connector area 20, in which the guide 26 is provided, becomes gradually thicker for this purpose. In this way, the springs 36 of the locking elements 33 are gradually tensioned during the movement from the first rotary position to the second rotary position, until they are relieved at the second rotary position when locking engagement with the recesses 27 takes place.

According to a possible embodiment, the connection between the sensor unit and the connector is splash-proof and/or watertight. This has the advantage that the patients/users can take a shower with it and that sweat would not cause any artifacts.

The housing may comprise a sealing element that interacts with a sealing surface of the connector. Preferably, the sealing element is pressed onto the sealing surface in the connected condition. This can be accomplished e.g. by a guide 26 extending such that an axial offset occurs along its extension in the circumferential direction, so that, in the second rotary position, the locking elements 33 will apply a force to the connector in an axial direction. The component used as a sealing surface may e.g. be the base plate 25 that interacts with a sealing element, e.g. a sealing ring, arranged on a housing edge.

The connector may be adhesively attached to the surface of the patch, the sealing surface of the connector surrounding the electrical connection area completely, so that the electrical connection area is fully sealed to the outside between the sensor unit and the patch.

Detaching the sensor unit 3 from the connector 2 after use is accomplished via operating elements 37, e.g. in the form of slide and/or toggle switches, which are positioned on the back of the sensor unit. The operating elements 37 are in contact with the locking elements 33 and, when actuated, they are able to wind up and/or tension the springs of the locking elements 33 again, thus retracting the locking elements 33. Only when both locking elements 33 are simultaneously retracted for more than 50% of their travel length will it be possible to remove them from the recesses 27, whereupon the sensor unit can be detached from the second rotary position in an axial direction and separated from the connector.

The locking elements 33 of the sensor unit may be located on the lower side of the sensor unit 3, embedded in sidewalls of a recess 32 in the housing of the sensor unit. The recess 32 may here correspond to the diameter and the height of the annular area 20 of the connector 2. In the connected condition, at least the annular area 20 of the connector 2 is located in the recess 32 of the housing. According to a possible embodiment, also the base plate 25 may be arranged countersunk in the recess 32, the latter being then provided with a step in a suitable manner.

The contact pins 35 are preferably arranged countersunk in the housing in an area 34 surrounded by the annular recess 32 and raised thereabove. At the end of the rotary movement and upon arriving at the second rotary position, the area 34 with the contact pins of the sensor unit comes to lie in the connector on the level of the patch, whereby the contact pins 35 will contact the contact areas 13 of the conductor tracks 12 of the patch.

Irrespectively of the exact structural design of the area 34 with the contact pins 35, the contact pins 35, in a first variant, extend beyond the lower edge of the housing 30, so as to make direct contact with the patch. In this case, the patch may be arranged flat on the lower surface of the connector in the area of contact areas 13, since the contact pins 35 extend fully through the recess or recesses 24 of the connector.

According to a second variant, however, an area of the patch with the contact areas 13 bulges into the recess or recesses 24 of the connector, e.g. by a raised area which is provided on the surface of the counter element and by means of which the contact areas are pressed into the recess 24 in the direction of the sensor unit. In this case, preferably only one recess 24 is provided for all the contact areas 13, so as to comply with the admissible bending radius of the patch. Due to the patch extending into the connector, it will suffice when the contact pins of the sensor unit project less far beyond the housing 30 of the sensor unit in order to contact the contact areas 13. In particular, the contact pins 35 according to the second version may not project beyond the lower edge of the housing 30, since they contact the contact areas 13 in a plane above the lower edge of the sensor unit.

A further possible design variant of the connector is a bayonet mount, which, comparable to the mounting of a lens of a reflex camera, is established by screwing in from a first rotary position to a second rotary position.

In addition, the sensor unit may comprise a magnetic component, which attracts a magnetic and/or metallic component located in the connector, whereby the sensor unit can be attached even more easily. This can be accomplished, for example, by a magnetically reacting metal alloy located in the connector ring.

1. A system for detecting biosignals, comprising a sensor unit and a body-attachable patch provided with electrodes and conductor tracks, the sensor unit and the patch being adapted to be mechanically connected to each other, via a connector arranged on the patch, in such a way that an electrical connection is established and the sensor unit is simultaneously held via the patch on the body, the sensor unit comprising a housing,
characterized in that
the sensor unit is releasably connectable to the connector by means of a rotation of the housing relative to the connector.

2. The system according to claim 1, wherein the connector comprises a mechanical connection area, which is releasably connectable to a mechanical connection area of the sensor unit by means of a rotary movement,
wherein, preferably, the mechanical connection areas are in locking engagement with each other in at least one defined rotary position and, further preferred, in only one defined rotary position,
and/or wherein, preferably, one of the mechanical connection areas comprises at least a one projecting annular segment and/or a substantially circular raised area and/or the other mechanical connection area comprises at least one annular groove segment and/or a substantially circular recess, the projecting annular segment and/or the substantially circular raised area being, in the connected condition, preferably accommodated, at least partially, in the annular groove segment and/or the substantially circular recess,
and/or wherein, preferably, the mechanical connection areas each surround an electrical connection area, the mechanical connection areas surrounding the electrical connection area preferably substantially circularly.

3. The system according to claim 2, wherein the mechanical connection areas are adapted to be pushed into one another in at least a first rotary position and to be moved, in the pushed-in condition, to a second rotary position by a rotary movement, the connection areas being locked on one another in the second rotary position,
and/or wherein one of the mechanical connection areas comprises at least one guide in which at least one locking element of the other mechanical connection area is guided during a rotary movement,
wherein the guide is preferably provided on the mechanical connection area of the connector, and/or wherein the guide is preferably configured as a groove in an outer or inner circumference of the mechanical connection area, the groove extending preferably in the circumferential direction,
and/or wherein the guide preferably has an recess into which the locking element snaps in position at the end of the rotary movement, the locking element being releasable from the snap-in position preferably against the force of a spring, the recess being preferably configured as a recess at the end of a groove forming the guide,
and/or wherein the mechanical connection area carrying the guide has an recess extending in the direction of the axis of rotation and allowing the locking element to be inserted into the guide.

4. The system according to claim 3, wherein the locking element is movable and preferably spring-loaded, and is in particular arranged on the sensor unit in a movable and preferably spring-loaded manner, wherein, preferably, at least one locking area of the locking element is movable in a radial direction relative to the axis of rotation, and/or wherein the locking element is preferably a displaceably supported pin and/or a rotatable hook and/or a rocker, wherein the direction of movement of the pin preferably extends in a radial direction relative to an axis of rotation of the connection between the connector and the sensor unit, and/or the axis of rotation of the hook and/or of the rocker preferably extends parallel to the axis of rotation of the connection between the connector and the sensor unit.

5. The system according to one of the claim 3 or 4, wherein the guide and/or the locking element are configured such that the torque to be created for the rotary movement increases at least over a subarea of the guide, in particular by reliably changing, in the closing direction, the distance between the axis of rotation of the connection between the connector and the sensor unit and the bottom of a groove serving as the guide, the torque preferably increasing continuously over at least 50% of the length of the guide.

6. The system according to one of the preceding claims, wherein the rotation of the housing takes place about an axis of rotation, which extends at an angle of less than 30° to a normal on the contact plane of the connector with the patch, preferably at an angle of less than 10°, and further preferred perpendicular to the contact plane of the connector with the patch, and/or wherein the rotation of the housing for connection between the sensor unit and the connector takes place through an angle of rotation between 20° and 180°, preferably through an angle of rotation between 40° and 90°.

7. The system according to one of the preceding claims, wherein the sensor unit has at least one operating element, by the actuation of which a locking engagement with the connector can be released, wherein, by actuating the operating element, the locking element can preferably be disengaged from the recess against the force of a spring, and/or wherein the operating element is preferably movably arranged on a housing of the sensor unit, in particular on a lateral or rear area of the housing, and/or wherein the operating element is preferably a component separate from the locking element, or wherein the operating element and the locking element are configured as an integral component.

8. A system, in particular a system according to one of the preceding claims, for detecting biosignals, comprising a sensor unit and a body-attachable patch provided with electrodes and conductor tracks, the sensor unit and the patch being adapted to be mechanically connected to each other, via a connector arranged on the patch, in such a way that an electrical connection is established and the sensor unit is simultaneously held via the patch on the body,
characterized in that
the connector establishes only the mechanical connection to the sensor unit and that electrical contacting takes place directly between the sensor unit and the patch.

9. The system according to claim 8, wherein the connector is shaped such that at least one contact surface of a conductor track of the patch is accessible from the sensor unit, wherein the connector preferably has an electrical connection area formed by at least one recess in the connector through which at least one contact surface of a conductor track of the patch is accessible, wherein preferably a plurality of contact surfaces are accessible through an recess of the connector and/or wherein preferably at least two contact surfaces of the patch are accessible through separate recesses of the connector,
and/or
wherein the patch side located opposite the connector has arranged thereon a counter element, which supports the patch in the area of the contact points, the counter element being preferably plate-shaped,
and/or wherein the patch has a bulge with which it extends into an recess of the connector, through which recess at least one contact surface of a conductor track of the patch is accessible from the sensor unit, wherein the bulge is created preferably via a raised area on a counter element arranged on the patch side facing away from the connector, the raised area pressing the patch into the opening.

10. The system according to one of the preceding claims, wherein the sensor element includes an electrical connection area comprising spring-loaded contact pins, which are used for electrically contacting the connector and/or the patch and which preferably enter directly into contact with contact surfaces of the conductor tracks of the patch, wherein the spring-loaded contact pins are arranged such that, in the contacted condition, they are preferably at least partially countersunk in the housing of the sensor unit and/or wherein, in the non-contacted condition, the spring-loaded contact pins preferably project beyond a lower edge of the housing of the sensor element.

11. The system according to one of the preceding claims, wherein the base area of the connector arranged on the patch is a maximum of 70% of the base area of the housing, preferably a maximum of 50%, further preferred a maximum of 30%, and/or wherein the mechanical connection with the connector is effected exclusively via a mechanical connection area arranged on the back of the housing, wherein lateral edges of the housing extend preferably at a distance from the mechanical connection area,
wherein the housing preferably tapers towards the connector on its back facing the patch, wherein, further preferred, the tapering area has a depth of at least 10% of the total depth of the housing, further preferred of at least 20% of the total depth and/or wherein the back of the housing facing the patch is preferably convex in shape.

12. The system according to one of the preceding claims, wherein the connector is arranged on a patch area configured as a folded flap,
wherein the connector is preferably movable relative to the non-folded part of the patch, at least a tilting movement being preferably possible between the connector and the non-folded part of the patch,
and/or
wherein, preferably, the flap is movable relative to the non-folded part of the patch, wherein, preferably, the flap is arranged on the patch in a freely folded manner or is flexibly fixed and/or point-fixed to an upper surface of the patch on at least one point of the latter, the fixing preferably leaving a distance between the upper surface of the non-folded part of the patch and the flap,
and/or
wherein, preferably, a counter element arranged on a flap side facing away from the connector is connected to the non-folded part of the patch,
and/or
wherein, preferably, the connector and/or the counter element comprise/comprises a web area around which the flap of the patch is guided in order to prevent a too small buckling radius of the patch,
and/or
wherein, preferably, the folded-over area, via which the flap is connected to the non-folded area of the patch, is wider than the base area of the connector and/or wherein the flap decreases in width from the folded-over area, via which it is connected to the non-folded area of the patch, towards its free end,
and/or
wherein, preferably, the flap is longer than the non-folded area of the patch, to which it is connected, so that a free end of the flap extends beyond an edge of the non-folded area of the patch, the non-folded area preferably extending in a widthwise direction in a band shape,
and/or
wherein, preferably, the conductor tracks of the patch are routed from the area of the connector via the flap to the electrodes, wherein, preferably, conductor tracks run from the flap in directions opposite to a non-folded, band-shaped area of the patch.

13. The system according to one of the preceding claims, wherein the connector is adhesively attached to the patch and/or wherein the patch is clamped in position between the connector and a counter element arranged on the patch side located opposite the connector.

14. A sensor unit for a system according to one of the preceding claims.

15. A connector or a patch with a connector for a system according to one of the preceding claims.
